(19) United States
(12) Patent Application Publication
(10) Pub. No.: US 2013/0109835 A1
(43) Pub. Date: May 2, 2013

(54) Method for processing protein-containing materials to obtain mixtures of natural amino acids, low-molecular weight peptides and oligopeptides

(71) Applicants: Victor S. Polyakov, Moscow (RU); Valeriy V. Ermilov, Moscow (RU); Dmitriy V. Kolotilin, Moscow (RU); Tatiyana Y. Fedorova, Moscow (RU)

(72) Inventors: Victor S. Polyakov, Moscow (RU); Valeriy V. Ermilov, Moscow (RU); Dmitriy V. Kolotilin, Moscow (RU); Tatiyana Y. Fedorova, Moscow (RU)

(73) Assignee: P.M.K. TECHNOLOGICAL TREATMENT LTD., Qiryat-Ono City (IL)

(21) Appl. No.: 13/146,662

(22) PCT Filed: Dec. 23, 2010
(86) PCT No.: PCT/RU10/00779

§ 371 Date: Jul. 28, 2011

(51) Int. Cl. C07K 1/12

(52) U.S. Cl. CPC C07K 1/12
USPC 530/343

(57) ABSTRACT

A method for processing protein-containing materials and utilization of protein-containing waste for production of mixtures of natural amino acids, mixtures of low-molecular weight peptides, and mixtures of oligopeptides, which can be used in the manufacture of reagents for the detoxification and bactericidal treatment of sewage sludge, surfactants, wetting agents, foaming agents, detergents and cleaners. The method includes processing protein-containing waste by hydrolysis with aqueous solutions of alkalis with additives in the form of ammonium salts of organic and mineral acids introduced into the reaction mixture of these components, and the wastes are processed in the mixture with water, alkali and additive, in the ratio of 1:(0.3-1.5):(0.03-0.45):(0.01-0.2), at 100-110° C. for 20-60 min.

### CROSS-REFERENCE TO RELATED APPLICATIONS

This application is a U.S. National Phase of PCT/RU2010/000779, filed on Dec. 23, 2010, which is incorporated by reference herein in its entirety.

### FIELD OF THE INVENTION

The invention relates to chemical technology and biotechnology, in particular, to methods for processing protein-containing materials and wastes in a mixture of natural amino acids, a mixture of low-molecular weight peptides and a mixture of oligopeptides, which can be used in the manufacture of reagents for detoxification and bactericidal treatment of sewage sludge, surfactants, wetting agents, foaming agents, detergents and cleaners, chelating compounds for combating chlorosis of plants, and micro fertilizers based on trace elements. Mixed compositions of protein hydrolysis products in concentrated form with a specified range of chain length, regulated concentration and completely preserved amino and carboxyl groups, are obtained.

### BACKGROUND OF THE INVENTION

A method of alkaline hydrolysis with the use of sodium hydroxide is known. The process is conducted at 95-115° C. by treating protein-containing raw materials with 8% aqueous solution of NaOH at atmospheric pressure for 8 hours (see SU 556776).

The disadvantage of this method is that it results in protein hydrolyzates of unknown composition with low and unregulated concentrations, and a long duration of the process.

A method of thermal hydrolysis of leather production waste with the addition of shale ash (an alkaline reagent—waste from the combustion of pyroshale) is known. The main stage of the hydrolysis lasts for 3-5 hours at 90-100° C. (see RU 2016521).

The disadvantage of this method is the incompleteness of protein hydrolysis, which requires additional stages of hydrolysis under more severe conditions, and the resulting hydrolyzates of indefinite composition with low yields.

Methods of utilizing animal protein containing waste by way of thermal hydrolysis with aqueous solutions of alkalis are also known (see SU 1496847, SU 1794089, RU 2021300).

However, these methods do not provide sufficient depth of animal protein hydrolysis, the compositions of the hydrolysis products vary widely, and there are impurities resulting from the cleavage of amino groups in deamination of amino acids and peptides mixtures.

A method of utilizing protein-containing waste when animal proteins, water and alkali are mixed in a ratio of 1:(1.8-2.0):(0.09-0.46) and are subjected to heat treatment with live steam at 120-180° C., is known. Depending on the ratio of the process parameters, a mixture of amino acids or their mixture with low-molecular weight peptides, or low-molecular weight peptides is obtained (see RU Patent 2291164). A similar pattern is used by the same authors in carrying out alkaline hydrolysis using live steam at 160-180° C. with a ratio of protein-containing waste, water and alkali of 1:(1.8-2.0):(0.38-0.46) and obtaining aqueous solutions of sodium or potassium salts of a mixture of amino acids (see patent RU 2282642) or with a ratio of the same components of 1:(1.8-2.0):(0.19-0.37) and obtaining aqueous solutions of sodium or potassium salts of amino acids or low-molecular weight peptides (see RU Patent 2,291,165).

The primary disadvantage of RU Patent 2291164 is low yields of sodium or potassium salts of a mixture of amino acids and peptides. During the alkaline hydrolysis of proteins at high temperatures of 120-180° C., deamination of the produced amino acids and peptides occurs, with the release of ammonia and formation of various carboxylic acids as impurities. Using live steam leads to uncontrolled local overheating of the reaction mass, with a significant deamination and decarboxylation of amino acids and peptides. In addition, the above conditions cause the destruction of certain amino acids, such as cysteine, cystine, serine, threonine (Lenindzher, A. Biochemistry. Moscow: Mir, 1974, 106.).

In these sources, at the indicated ratios of protein/water/alkali, a low concentration of alkaline solutions of 4.5-20% is used, since increased concentration of alkaline solutions at such high temperatures leads to increased deamination of amino acids and peptides. Using low concentrations of alkaline solutions does not result in mixtures of amino acids or peptides with high concentrations. Theoretically, the maximum possible concentrations of amino acid mixture at the indicated ratios of initial reagents are 1.5-1.7 mol/L, assuming the protein content in protein-containing waste is 100% and all amino groups are fully retained. Due to the deamination and degradation occurring at the local superheating by live steam and uncontrolled dilution of the reaction mass by condensate, the concentration of amino acids mixtures is, in practice, much lower.

Thus, low uncontrolled yields of amino acids and peptides mixtures, a large amount of impurities resulting from degradation of amino acid fragments, do not allow obtaining high-quality reagents for detoxification and bactericidal treatment of sewage sludge, and other quality products based on them.

### DETAILED DESCRIPTION OF THE PREFERRED EMBODIMENTS

The problem to be solved by the process described herein is multifaceted; namely, the first aspect of the problem can be identified as the problem of developing environmentally safe recycling of protein from protein-containing waste in a mixture of natural amino acids, low-molecular weight peptides, peptides with an average chain length and oligopeptides with high yields and selectivity of the process. The second aspect of the problem is the maximum possible preservation of the structures of amino acids and peptides during thermal alkaline hydrolysis of protein, the possibility of obtaining narrow groups of compositions with a specified range of the peptide chain length. The third aspect of the problem is the development of a method for regulating the concentration of natural amino acids or peptides mixtures and the possibility of achieving high concentrations of resulting compositions.

The solution of this multifaceted problem is achieved by subjecting protein-containing waste to alkaline hydrolysis at 100-110° C. in the presence of special additives introduced into the reaction mixture to prevent the degradation of amino groups. The use of highly concentrated solutions of alkali in this case allows obtaining a reaction mass with a high concentration of hydrolysis products. The high selectivity of the process and high yield of products due to the absence of deamination and degradation processes allow obtaining rather narrow ranges of hydrolysis products by the peptide chain length resulted from varying the ratio of initial components. The process conditions, under which all the pathogenic microflora in protein-containing wastes undergoing the initial processes of putrefaction, fermentation and moldy formation is destroyed, allow obtaining environmentally safe products of natural origin (a mixture of amino acids and peptides) without impurity of degradation products.

Technical results from the use of the invention include environmentally safe processing of protein-containing wastes, while obtaining mixtures of natural amino acids or peptides of the specified composition with high yields and required concentration, as marketable products.

The above technical results are achieved by introduction of special additives into the reaction mixture during the hydrolysis with aqueous solutions of alkalis while the wastes are processed in the mixture with water, alkali and additive in a ratio of 1:(0.3-1.5):(0.03-0.45):(0.01-0.2) at 100-110° C. for 20-60 min.

The composition of hydrolysis products can be regulated by changing the process parameters as follows:

for obtaining a mixture of alkali metal salts and oligopeptides, the ratio of waste, water, alkali and additive should be 1:(0.3-0.9):(0.03-0.07):(0.01-0.03) with the process duration of 35-60 min;

for obtaining a mixture of alkali metal salts and penta-decapeptides, the ratio of waste, water, alkali and additive should be 1:(0.4-1.1):(0.08-0.15):(0.01-0.07) with the process duration of 35-55 min;

for obtaining a mixture of alkali metal salts and low-molecular weight peptides, the ratio of waste, water, alkali and additive should be 1:(0.6-1.3):(0.16-0.29):(0.01-0.14) with the process duration of 25-50 min;

for obtaining a mixture of alkali metal salts and natural amino acids, the ratio of waste, water, alkali and additive should be 1:(1.0-1.5):(0.30-0.45):(0.01-0.2) with the process duration of 20-45 min.

Additives preventing deamination of formed products are added into the reaction mixture, consisting of a protein-containing waste, water and alkali in a specified ratio. This provides the extraction of a greater amount of individual amino acids and peptides, and increases their yield and process selectivity. The simultaneous reduction of the process temperature down to 100-110° C. allows the use of more concentrated solutions of alkaline agents and obtaining amino acid and peptide compositions of high concentration. The described process conditions allow obtaining narrow composition groups of mixtures of oligopeptides or penta-decapeptides or low-molecular (di-tetra) weight peptides or amino acids with high yields by changing specified process parameters.

The following wastes can be used as raw materials for the process: leather industry wastes (raw scrapings, raw hide, chrome shavings, ashes), fur production wastes, poultry farms wastes (down, feather, feather meal), cloth production wastes, felt boots production wastes (fringes, trimmings), food and dairy industries wastes, casein, and meat and poultry production wastes.

Sodium hydroxide or potassium hydroxide can be used as the alkaline agent.

Ammonium salts of organic and mineral acids can be used as the additives. The additives may be selected from the group of the following compounds: ammonium acetate, ammonium citrate, ammonium chloride, ammonium sulfate, ammonium bicarbonate, monosodium phosphate, twice-substituted ammonium phosphate, and other ammonium salts.

The following examples further illustrate the proposed method. These typical examples of obtaining specific compositions according to the claimed invention do not restrict in any way the scope of its legal protection. The examples only show a concrete illustration of the proposed method.

### Example 1

The feedstock is raw scrapings, leather production wastes. 70 liters of tap water are fed into a 450 L reactor equipped with an anchor stirrer, reflux condenser and a jacket for heating with indirect heating vapor. Then the reactor is turned on, and 36.3 kg of sodium are loaded in portions. The solution temperature rises up to 62° C. due to dissolution of the alkali. Chunks of scrapings in the amount of 80.7 kg are moisturized and homogenized with a solution of 810 g of ammonium chloride in 10 L of water and are fed into the reactor as a compacted mass. The ratio of protein-containing waste, water, alkali and ammonium salt is 1:1.0:0.45:0.01. Then the steam supply is turned on, and the temperature rises up to 105° C. The reaction mixture is stirred for 20 minutes, then the steam is turned off and the unit is cooled down to 35-40° C. The reaction mass is extracted, and 180 L of a mixture of sodium salts of natural amino acids are obtained. The hydrolyzate comprises the following amino acids (wt %):

glycine—12.4;

alanine—9.6;

valine—2.7;

leucine—4.6;

isoleucine—1.7;

threonine—1.9;

phenylalanine—1.6;

tyrosine—2.7;

proline—8.6;

serene—3.5;

aspartic acid—7.2;

glutamic acid—10.1;

histidine—1.8;

lysine—5.4;

arginine—6.5.

The content of amino groups in the hydrolyzate, determined by the Van Slyke method and translated to amino acids' concentration based on the average molecular weight of the mixture of amino acids, is 3.51 mol/L. Potentiometric titration with 1 M HCl showed that the concentration of amino acids was 3.62 mol/L and the content of free alkali was 0.07 mol/L. The yield of amino acids mixture, translated to protein content in the feedstock, is about 98%.

### Example 2

A composition of a mixture of sodium salts of natural amino acids is obtained similar to Example 1, except that the hydration and homogenization of scrapings is carried out in 10 liters of water without the addition of NH₄Cl. 180 L of a mixture of sodium salts of natural amino acids are obtained. The content of amino groups in the hydrolyzate, translated to amino acid concentration, is 2.66 mol/L, the content of free alkali is 0.06 mol/L. The yield of a mixture of amino acids, translated into the protein content in the feedstock, is 74.3%.

### Example 3

The feedstock is chrome shavings and raw hide trimmings, leather production wastes. 50 L of water are fed into the reactor, described in Example 1, then agitation is turned on, and 20.1 kg of sodium hydroxide are loaded in portions. The solution temperature rises to 54° C. due to dissolution of the alkali. Trimmings in the amount of 34.3 kg and chrome shavings in the amount of 32.8 kg are moisturized and homogenized with a solution of 13.4 kg of ammonium bicarbonate in 55 L of water and are fed into the reactor as a compacted mass. The ratio of protein-containing waste, water, alkali and ammonium salt is 1:1.5:0.3:0.2. Then the steam supply is turned on, and the temperature rises to 100° C. The reaction mixture is stirred for 45 minutes, then the steam is turned off and the unit is cooled down to 35-40° C. The reaction mass is extracted, and 180 L of a mixture of sodium salts of natural amino acids are obtained. The content of amino groups in the hydrolyzate, translated to amino acids concentration, is 1.82 mol/L, the content of free alkali is 0.03 mol/L.

### Example 4

The feedstock is raw hide trimmings, moldy and rotting hide, waste from tanneries. 37 L of water are fed into the reactor, described in Example 1, then agitation is turned on, and 6.7 kg of sodium hydroxide are loaded in portions. The solution temperature rises up to 54° C. due to dissolution of the alkali. Trimmings in the amount of 48.5 kg and moldy and rotting hide in the amount of 43.8 kg are moisturized and homogenized with a solution of 930 g of ammonium sulfate in 19 L of water and are fed into the reactor as a compacted mass. The ratio of protein-containing waste, water, alkali and ammonium salt is 1:0.6:0.29:0.01. Then the steam supply is turned on, and the temperature rises to 106° C. The reaction mixture is stirred for 25 minutes, then the steam is turned off and the unit is cooled down to 35-40° C. The reaction mass is extracted, and 160 L of a mixture of sodium salts of low-molecular (di-tetra) weight peptides are obtained. The content of amino groups in the hydrolyzate, translated to peptides concentration, is 1.4 mol/L, the content of free alkali is 0.09 mol/L.

### Example 5

The feedstock is scraps of fur production and waste after hides tanning. 2.5 L of water are fed into a 15 L reactor, equipped with a frame stirrer, reflux condenser and steam jacket, then agitation is turned on, and 560 g of sodium hydroxide are loaded in portions. The solution temperature rises up to 42° C. due to dissolution of the alkali. The waste in the amount of 3.5 kg is moisturized and homogenized with a solution of 490 g of ammonium acetate in 2 L of water and is fed into the reactor as a compacted mass. The ratio of protein-containing waste, water, alkali and ammonium salt is 1:1.3:0.16:0.14. Then the steam supply is turned on, and the temperature rises to 102° C. The reaction mixture is stirred for 50 minutes, then the steam is turned off, and the unit is cooled down to 35-40° C. The reaction mass is extracted, and 8.3 L of a mixture of sodium salts of low-molecular (di-tetra) weight peptides are obtained. The content of amino groups in the hydrolyzate, translated to peptides concentration, is 1.1 mol/L, the content of free alkali is 0.03 mol/L.

### Example 6

The feedstock is tow, parings and other waste products of felting. 1.2 L of water are fed into the reactor described in Example 5, then agitation is turned on, and 870 g of sodium hydroxide are loaded in portions. The solution temperature rises up to 65° C. due to dissolution of the alkali. The waste in the amount of 5.8 kg is moisturized and homogenized with a solution of 58 g of ammonium chloride in 1.1 L of water and is fed into the reactor as a compacted mass. The ratio of protein-containing waste, water, alkali and ammonium salt is 1:0.4:0.15:0.01. Then the steam supply is turned on, and the temperature rises to 108° C. The reaction mixture is stirred for 30 minutes, then the steam is turned off, and the unit is cooled down to 35-40° C. The reaction mass is extracted, and 8.25 L of a mixture of sodium salts of penta-decapeptides are obtained. The content of amino groups in the hydrolyzate, translated to peptides concentration, is 0.7 mol/L, the content of free alkali is 0.08 mol/L.

### Example 7

The feedstock is waste from technical casein production. 3.2 L of water are fed into the reactor described in Example 5, then agitation is turned on, and 380 g of sodium hydroxide are loaded in portions. The solution temperature rises up to 38° C. due to dissolution of the alkali. The waste in the amount of 4.7 kg is moisturized and homogenized with a solution of 320 g of ammonium bicarbonate in 2 L of water and is fed into the reactor as a compacted mass. The ratio of protein-containing waste, water, alkali and ammonium salt is 1:1.1:0.08:0.07. Then the steam supply is turned on, and the temperature is risen up to 103° C. The reaction mixture is stirred for 55 minutes, then the steam is turned off, and the unit is cooled down to 35-40° C. The reaction mass is extracted, and 9.9 L of a mixture of sodium salts of peptides are obtained. The content of amino groups in the hydrolyzate, translated to peptides concentration, is 0.95 mol/L, the content of free alkali is 0.01 mol/L.

### Example 8

The feedstock is chicken feathers waste of poultry farms. 340 mL of water are fed into the reactor described in Example 5, then agitation is turned on, and 245 g of sodium hydroxide are loaded in portions. The solution temperature rises up to 62° C. due to dissolution of the alkali. The waste in the amount of 3.5 kg is moisturized and homogenized with a solution of 35 g of disubstituted ammonium phosphate in 760 mL of water and is fed into the reactor as a compacted mass. The ratio of protein-containing waste, water, alkali and ammonium salt is 1:0.3:0.07:0.01. Then the steam supply is turned on, and the temperature rises to 110° C. The reaction mixture is stirred for 35 minutes, then the steam is turned off, and the unit is cooled down to 35-40° C. The reaction mass is extracted, and 4.4 L of a mixture of sodium salts of oligopeptides is obtained. The content of amino groups in the hydrolyzate, translated to peptides concentration, is 0.51 mol/L, the content of free alkali is 0.06 mol/L.

### Example 9

The feedstock is chicken feathers waste of poultry farms. 200 mL of water are fed into the reactor described in Example 5, then agitation is turned on, and 96 g of sodium hydroxide are loaded in portions. The solution temperature rises up to 50° C. due to dissolution of the alkali. The waste in the amount of 3.2 kg is moisturized and homogenized with a solution of 96 g of ammonium bicarbonate in 2.7 L of water and is fed into the reactor as a compacted mass. The ratio of protein-containing waste, water, alkali and ammonium salt is 1:0.9:0.03:0.03. Then the steam supply is turned on, and the temperature rises to 104° C. The reaction mixture is stirred for 35-40 minutes, then the steam is turned off, and the unit is cooled down to 35-40° C. The reaction mass is extracted, and 5.8 L of a mixture of sodium salts of oligopeptides are obtained. The content of amino groups in the hydrolyzate, translated to peptides concentration, is 0.4 mol/L, the content of free alkali is 0.01 mol/L.

The toxicity of the resulting mixtures of natural amino acids and mixtures of peptides in enteral administration of LD₅₀ exceeds 20 g/kg, which allows to assign them to the class of low hazard substances.

Thus, the above examples confirm the feasibility of the invention and the claimed benefits of the proposed method of processing protein-containing wastes with obtaining environmentally friendly mixtures of natural amino acids or peptides with a specified range of chain length.

The industrial applicability of the claimed method is also confirmed by the foregoing embodiments. It should be noted that the claimed method of processing protein-containing waste allows selective obtaining of groups of natural amino acids or peptides with a specified chain length used for production of a specific type of products. Simultaneously, the proposed method allows obtaining environmentally safe amino acid or peptide compositions of high concentration, which is economically expedient.

Having thus described a preferred embodiment, it should be apparent to those skilled in the art that certain advantages of the described method and apparatus have been achieved.

It should also be appreciated that various modifications, adaptations and alternative embodiments thereof may be made within the scope and spirit of the present invention. The invention is further defined by the following claims.

What is claimed is:

1. A method for processing protein-containing waste by hydrolysis with aqueous solutions of alkalis, comprising:
introducing additives in a form of ammonium salts of organic and mineral acids into a reaction mixture of the protein-containing waste;
agitating the waste in the mixture with water, an alkaline agent and the additive, in a ratio of 1:(0.3-1.5):(0.03-0.45):(0.01-0.2) at a temperature of 100-110° C. for 20-60 min.

2. The method of claim 1, wherein the alkaline agent is sodium hydroxide.

3. The method of claim 1, wherein the alkaline agent is potassium hydroxide.

4. The method of claim 1, wherein the processing of the protein-containing waste mixed with the water, the alkaline agent and the additive, in a ratio of 1:(1.0-1.5):(0.30-0.45):(0.01-0.2), is carried out at 100-110° C. for 20-45 min yielding a mixture of alkali metal salts of natural amino acids.

5. The method of claim 1, wherein the processing of the protein-containing waste mixed with the water, the alkaline agent and the additive, in a ratio of 1:(0.6-1.3):(0.16-0.29):(0.01-0.14), is carried out at 100-110° C. for 25-50 min, yielding the hydrolysis product as a mixture of alkali metal salts of low-molecular weight peptides.

6. The method of claim 1, wherein the processing of the protein-containing waste mixed with the water, the alkaline agent and the additive, in a ratio of 1:(0.4-1.1):(0.08-0.15): (0.01-0.07), is carried out at 100-110° C. for 30-55 min yielding the hydrolysis product as a mixture of alkali metal salts of penta-decapeptides.

7. The method of claim 1, wherein the processing of the protein-containing waste mixed with the water, the alkaline agent and the additive, in a ratio of 1:(0.3-0.9):(0.03-0.07):(0.01-0.03), is carried out at 100-110° C. for 35-60 min, yielding the hydrolysis product as a mixture of alkali metal salts of oligopeptides.
